(19) United States
(12) Patent Application Publication
(10) Pub. No.: US 2026/0008988 A1
(43) Pub. Date: Jan. 8, 2026

(54) Ergonomic Manual Colony Counter

(71) Applicant: INTERSCIENCE, St. Nom la Breteche (FR)

(72) Inventor: Emmanuel JALENQUES, St Nom la Breteche (FR)

(21) Appl. No.: 18/992,898

(22) PCT Filed: Sep. 18, 2023
(86) PCT No.: PCT/EP2023/075651

§ 371 Date: Jan. 9, 2025

(30) Foreign Application Priority Data
Sep. 19, 2022 (FR) FR2209407

(51) Int. Cl. C12M 1/34; C12M 1/00; C12M 1/12; C12M 1/22

(52) U.S. Cl. CPC C12M 41/36; C12M 23/10; C12M 23/26; C12M 23/52; C12M 25/00

(57) ABSTRACT

Manual colony counter (1) comprising:—a face (2), referred to as the functional face, said functional face comprising a zone (3), referred to as the counting zone, on which is intended to rest a culture support for growing microorganisms that are intended to be counted manually,—a mobile part (4), movable relative to the functional face and designed to be arranged in at least two different positions: ● a position referred to as the deployed position (6), in which a surface of the mobile part forms a bearing surface (41) for the hand and/or the wrist and/or the forearm and/or the elbow of the user, and ● a position referred to as the stowed position (5).

### TECHNICAL AREA

The present invention relates to a counter for the manual counting of microorganisms and, in particular, for the manual counting of bacterial colonies.

The present invention finds applications in the field of research and the agro-food, medical, cosmetic and pharmaceutical industries.

### STATE OF THE PRIOR ART

Manual colony counters are known from the state of the art.

State-of-the-art colony counters include a tactile zone on which, usually, a Petri dish, comprising an agar on the surface of which the seeding and then the incubation have previously been carried out, is placed. The user will, by means of a stylus or a pen, exert pressure on each observable colony and will, generally, simultaneously mark the identified colony. Generally, gripping the stylus or pen may require that, during the counting/marking operation, the operator's hand with which he carries out the counting is supported near the Petri dish. In addition, during the counting operation, the operator must adopt a particular position, relative to the Petri dish, so that the area of the Petri dish observed is intersected by the visual axis. This particular position is not universal and common to all colony counters but it depends on the shape and arrangement of the counter and, in particular, on the arrangement of the lighting device of the touch zone. Colony counters also include a counting function which increments each pressure exerted by the user, and therefore each colony identified, so that the total number of colonies contained in the analyzed Petri dish is determined.

A recurring problem with state-of-the-art colony counters lies in the fact that the user in charge of counting must carry out this counting operation on a substantial number of samples successively. Also, discomfort is quickly felt by the user due to the posture which is imposed on the user.

An aim of the invention is, furthermore, to propose a manual colony counter:
whose ergonomics are optimized, and/or
making it possible to reduce, or even eliminate, the discomfort felt by the user during counting and/or to prevent musculoskeletal disorders.

### PRESENTATION OF THE INVENTION

For this purpose, a manual colony counter is proposed comprising a face, called a functional face, said functional face comprises a zone, called counting zone, on which, and/or in which, is intended to rest a culture support of microorganisms, preferably bacteria, intended to be counted manually.

The manual colony counter comprises a movable part, relative to the functional face, arranged to be arranged in at least two different positions:
a position, called deployed, in which a surface of the moving part forms a support surface for the hand and/or wrist and/or forearm and/or elbow of the user, and
a position, called retired.

Preferably, the colony counter comprises an optical device arranged to backlight the culture support.

Preferably, the counter may comprise or be arranged to communicate with a device arranged to increment each pressure exerted on the touch zone.

Preferably, the zone on which the microorganism culture support is intended to rest comprises the device arranged to increment each pressure exerted on the tactile zone.

Preferably, the area on which the microorganism culture support is intended to rest is a tactile area.

The counter may not include a device designed to increment each pressure exerted on the touch zone.

The touch zone may be a touch surface.

The microorganism culture support is, by way of non-limiting example, a Petri dish.

Preferably, the counter may include an additional support surface on the functional face. The additional support surface can serve as support for the user's hand.

Preferably, the movable part is arranged to be moved relative to the functional face and/or relative to the existing additional support surface of the functional face.

Preferably, the support surface is adjacent or contiguous to the counting zone.

Preferably, in the deployed position, the support surface can constitute an extension of the additional support surface of the functional face.

Preferably, in the deployed position, the support surface and/or the moving part:
extends beyond the functional side, and/or
is protruding from the functional face, and/or
forms a surface or element distinct from the functional face.

Preferably, in the folded position, the movable part does not offer a support surface to the user and, in particular, a support surface for the hand and/or wrist and/or forearm and/or the user's elbow.

Preferably, the pressure is exerted on the microorganism support which exerts, by reaction, pressure on the counting zone.

Preferably, the functional face extends mainly along a plane.

Preferably, during counting, the moving part is in the deployed position.

Preferably, the deployed position allows better comfort of use of the colony counter for the user.

Preferably, when the colony counter is not used, the moving part is in the folded position.

Preferably, the folded position allows the colony counter to be more compact or less bulky to facilitate storage and/or reduce bulk.

Preferably, the surface of the moving part forming the support surface is an upper surface of the moving part.

Preferably, the counting zone is capable of and/or arranged to allow lighting or backlighting of the culture support.

Preferably, the counting zone is able to, preferably still arranged to, receive, more preferably to cooperate with, the culture support.

Preferably, when the movable part is in the deployed position, an axis connecting a center of the support surface to a center of the counting zone, called the support axis, forms an angle À between 20° and 70°, preferably between 35° and 55°, relative to a median axis extending between a side or a face of the functional face intended to be facing the user, called the proximal side, and the center of the counting zone.

The angle formed between the support axis and the central axis can be a positive angle or a negative angle. Depending on the clockwise direction, the angle formed between the support axis and the central axis can be a positive angle, preferably for right-handed users, and can be a negative angle, preferably for left-handed users.

Preferably, the central axis extends equidistant from two lateral sides of the colony counter.

Preferably, the two lateral sides of the colony counter each connect the proximal side of the colony counter to a side, called the distal side, of the colony counter which is opposite the proximal side.

Preferably, the functional face and the movable part, preferably the bearing surface of the movable part, preferably in the deployed position and/or in the retracted position, form an angle less than or equal to 45° between them, preferably are parallel between them.

Preferably, a plane along which the functional face and/or the additional support surface of the functional face mainly extends, called the plane of the functional face, and a plane in which the movable part mainly extends, called the plane of the movable part, preferably in the deployed position and/or folded position, form an angle of less than 20° between them, preferably are parallel to each other.

Preferably, the plane of the functional face and the plane of the movable part forms, preferably the plane of the support surface, preferably in the deployed position and/or folded position, are included in the same plane or are included in two planes combined.

Preferably, the functional face and the moving part, preferably the bearing surface of the moving part, preferably in the deployed position and/or folded position, form an angle less than or equal to 20° between them.

Preferably, the functional face forms an angle of between 12.5° and 27.5° relative to a face, preferably a lower face, of the meter on which the latter is intended to rest.

Preferably, the four sides of the colony counter, that is to say the two lateral sides and the proximal and distal sides, connect the lower face of the colony counter to the functional face.

Preferably, the angle formed between the functional face and the lower face of the colony counter is greater than or equal to 12.5°, preferably 15°, more preferably 16°, advantageously 17°, preferably at 18°, even more preferably at 19° and most preferably at 20°.

Preferably, the angle formed between the functional face and the lower face of the colony counter is less than or equal to 27.5°, preferably 25°, more preferably 24°, advantageously 23°, preferably at 22°, even more preferably at 21° and most preferably at 20°.

Preferably, the counting zone and/or the functional face comprises:
a recess (31) comprising a bottom (8) on which the microorganism culture support is intended to rest, or
an orifice comprising, on its periphery, one or more gripping means on which is intended to rest the microorganism culture support.

Preferably, the gripping means(s) are capable and/or arranged to receive and/or support and/or retain and/or maintain the microorganism culture support.

Preferably, the gripping means(s) form or constitute the device arranged to increment each pressure exerted.

Preferably, the recess and the bottom form a housing capable of receiving and/or arranged to receive and/or in which the culture support is intended to rest.

Preferably, the orifice and the gripping means(s) form a housing capable of receiving and/or arranged to receive and/or support and/or retain and/or maintain the culture support and/or in which is intended to rest the culture medium.

Preferably, the recess is or includes a notch.

Preferably, the tactile zone forms the bottom of the recess.

Preferably, the recess is intended to cooperate with the culture support.

Preferably, the recess has a shape capable of receiving growing media.

Preferably, when the movable part is in the folded position, at least a part, preferably the whole, of said movable part covers the counting zone and/or the recess and/or the notch and/or the orifice.

Preferably, the covering, by the moving part, of the counting zone makes it possible to protect the colony counter, for example, from shocks, liquid splashes or even dust.

Preferably, the movable part is arranged to be translated along the functional face or along a side adjoining the functional face.

Preferably, the movable part or part of the movable part arranged to be translated along the functional face or along a side adjoining the functional face can form a cover for the functional zone.

Preferably, the movable part is arranged to pivot around an axis of rotation parallel to the functional face or around an axis of rotation perpendicular to the functional face.

The movable part, to ensure the passage from the retracted position to the deployed position, and vice versa, can be retractable and/or foldable and/or be pivoted or translated.

Preferably, the colony counter comprises a control unit arranged to:
activate the colony counter in response to a passage of the moving part from the retracted position to the deployed position, and/or
deactivate the colony counter in response to a passage of the moving part from the deployed position to the retracted position.

Activation of the colony counter can mean a transition from an operating state to an inactive or deactivated state. It can be understood activating the colony counter can be a transition from an inactive or deactivated state to a working state. Preferably, the control unit can be arranged to activate and/or deactivate the colony counter.

Preferably, the colony counter, preferably the control unit, can be arranged to defer the activation and/or inactivation of the colony counter for a period of time. In other words, a period of time may separate the passage of the moving part from the retracted position to the deployed position, or vice versa, from the activation, or respectively the inactivation, of the colony counter.

Preferably, the additional bearing surface of the functional face and/or the bearing surface of the moving part is formed by an elastic material.

Preferably, the elastic material is a polymer, more preferably an elastomer.

Preferably, the elastic material is a foam.

Preferably, the elastic material is neoprene or ethylene-propylene-diene monomer, denoted EPDM.

Preferably, the additional bearing surface of the functional face and/or the bearing surface of the moving part is smooth or has low roughness or is covered by a non-porous material.

Preferably, the microorganism culture support intended to rest on the counting zone is a Petri dish.

The colony counter may include one or more adapters, preferably removable, arranged to cooperate with the counting zone and intended to:
be inserted or introduced into the counting zone, and
receive culture support for microorganisms intended to be counted manually.

For each pressure exerted on the touch zone, the colony counter, preferably the control unit, can be arranged to:
modulate, preferably briefly, a light intensity emitted by the optical device arranged to backlight the culture support,
emit a sound signal.

Preferably, the sound signal is emitted by an audio device of the colony counter, by way of non-limiting example, a loudspeaker. The colony counter may comprise one or more optical filters intended and/or arranged to be arranged on the counting zone, the optical filter(s) are arranged to modulate or modify the contrast between the colonies and a culture medium in which the colonies incubate.

### DESCRIPTION OF FIGURES

Other advantages and particularities of the invention will appear on reading the detailed description of implementations and embodiments in no way limiting, and the following appended drawings: FIG. 1a is a schematic representation in oblique view of a first embodiment of the colony counter according to the invention,

FIG. 1b is a schematic representation in side view of the first embodiment of the colony counter according to the invention,

FIG. 2a is a schematic representation in front view of the first embodiment of the colony counter according to the invention in which the movable part is in the folded position,

FIG. 2b is a schematic representation in front view of the first embodiment of the colony counter according to the invention in which the part mobile is in the deployed position,

FIG. 3 is a schematic representation in side view of a second embodiment of the colony counter according to the invention,

FIG. 4a is a schematic representation in front view of the second embodiment of the colony counter according to the invention in which the movable part is in the folded position,

FIG. 4b is a schematic representation in front view of the second embodiment of the colony counter according to the invention in which the movable part is in the deployed position,

FIG. 5 is a schematic representation in front view of a third embodiment of the colony counter according to the invention in which the movable part is in the folded position,

FIG. 6 is a schematic representation in front view of the third embodiment of the colony counter according to the invention in which the movable part is in the deployed position.

### DESCRIPTION OF EMBODIMENTS

The embodiments described below being in no way limiting, we may in particular consider variants of the invention comprising only a selection of characteristics described, isolated from the other characteristics described (even if this selection is isolated within a sentence including these other characteristics), if this selection of characteristics is sufficient to confer a technical advantage or to differentiate the invention compared to the state of the prior art. This selection includes at least one characteristic, preferably functional without structural details, or with only part of the structural details if this part only is sufficient to confer a technical advantage or to differentiate the invention compared to the state of the prior art.

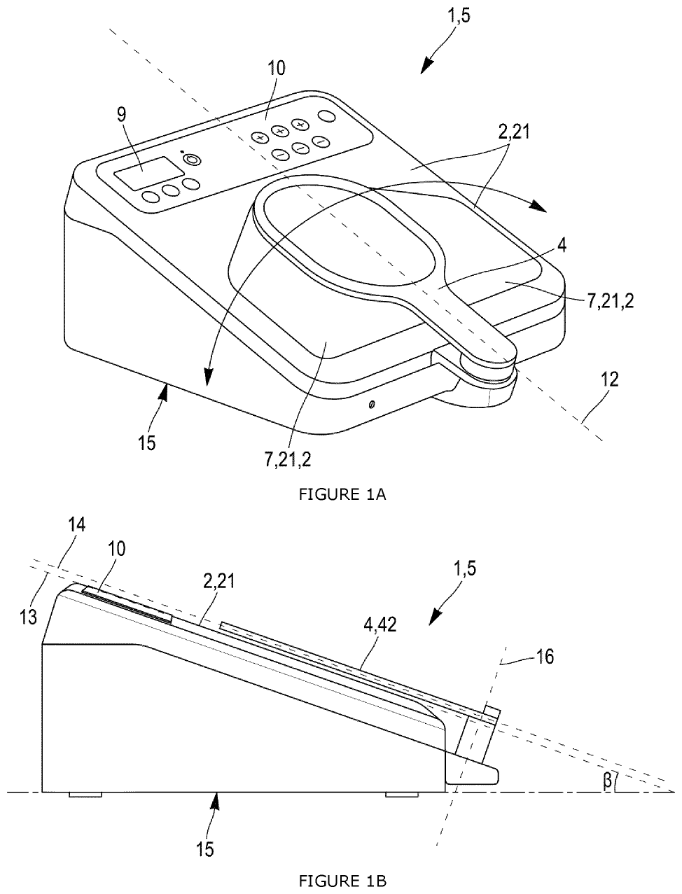

With reference to FIGS. 1 to 6, three embodiments of the manual colony counter 1 according to the invention are presented. In the absence of indications to the contrary, the characteristics described are common to each of the embodiments.

The colony counter 1 comprises a functional face 2. The functional face 2 comprises a counting zone 3 on which a culture support for microorganisms intended to be counted manually is intended to rest. The counting zone 3 is able to allow the backlighting of the culture support. In the majority of cases, the culture medium is a Petri dish.

The colony counter I also includes a lighting device arranged to backlight the counting zone 3.

The luminous flux emitted by the lighting device is adjustable.

The colony counter 1 further comprises a movable part 4 relative to the functional face 2. The movable part 4 is arranged to be arranged in at least two different positions. A position 6 is called deployed position 6. In deployed position 6, an upper surface of the movable part 4 forms a support surface 41 for the user's forearm and elbow. The support surface can be formed by the entire upper surface 41 of the movable part 4. Another of the positions is called the folded position 5. In the folded position 5, the movable part 4 is not arranged so to form a support surface 41 for the user's forearm and elbow.

According to the embodiments presented, the functional face 2 comprises a support surface 7, called the additional support surface 7. The additional support surface 7 adjoins the counting zone 3. The additional support surface 7 serves as a hand rest for the user. In the deployed position 6, the user can therefore rest his hand and his arm during counting, like the position adopted for writing with a pen. According to the embodiment, and in a non-limiting manner, the support surface 41 and the additional support surface 7 are made of elastomer.

According to the embodiments presented, and in a non-limiting manner, the counting zone 3 comprises a recess 31. The bottom 8 of the recess 31 is formed by a transparent surface 8. The microorganism culture support is intended to rest on the transparent surface 8. The transparent surface 8 is a tactile surface arranged to increment each pressure exerted on the transparent surface 8 by the user.

According to the embodiments presented, the functional surface 2 comprises a screen 9 on which the incremented number of colonies appears. According to the embodiment, the functional face 2 further comprises a control panel 10. The control panel includes controls making it possible to adjust certain parameters of the meter such as, for example, the volume of the sound signal emitted by the meter during an increment and/or the type of sound signal emitted and/or the sensitivity of a sensor arranged to detect pressure exerted on the tactile area and/or a change in light intensity.

According to the embodiments presented, and with reference to FIG. 1b, the upper surface of the functional face 2 forms an angle B between 12.5° and 27.5° relative to the face 15 of the colony counter 1 on which the latter is intended to rest. This allows the user to perform the count in a position in which the elbow is relaxed. According to the embodiment, and in a non-limiting manner, the angle β is 20°.

According to the embodiments presented, when the moving part 2 is in the deployed position 6, the axis 11 connecting a center of the support surface 41 to a center of the counting zone 3 forms an angle À between 20° and 70° relative to the median axis 12 extending between the side, called the proximal side, of the functional face 2 intended to be facing the user and the center of the counting zone 3. The support 41 is sufficiently large so that the user has sufficient latitude to position his forearm so that it forms an angle, relative to the median axis 12, which suits him and which is between 20° and 70°.

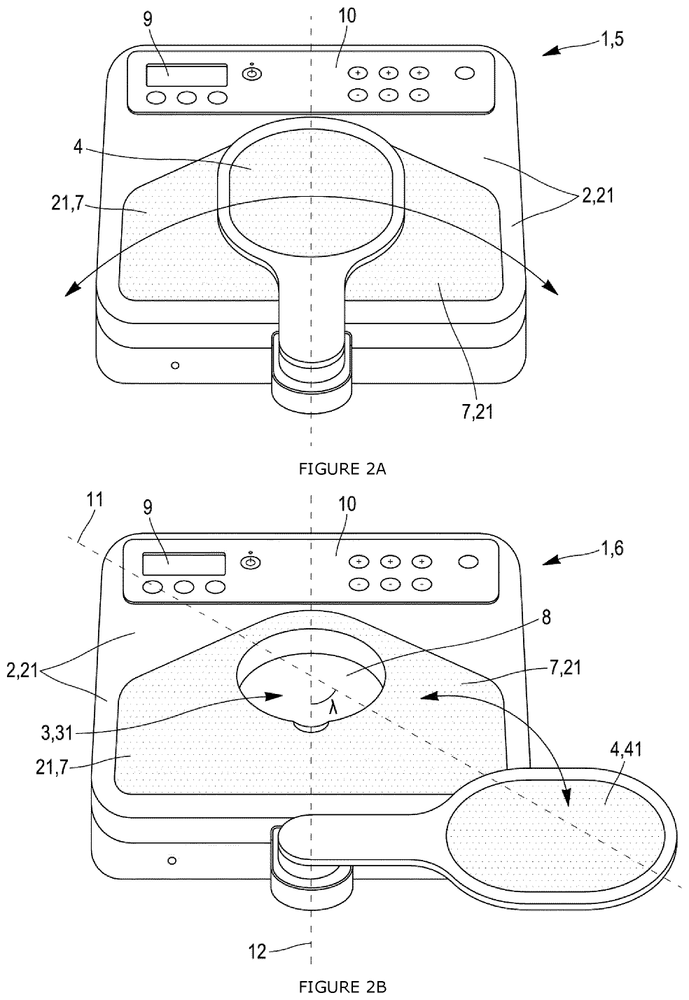

According to the first embodiment, and with reference to FIGS. 1 and 2a, the movable part 4 is arranged to pivot around an axis of rotation 16 perpendicular to the upper surface 21 of the functional face 2. Furthermore, in the position folded 5, the moving part 4 covers the counting zone 3. Also, in the folded position 5, the moving part 4 is therefore used as a cover protecting the counting zone 3. The colony counter I according to the first embodiment is suitable for both left-handed and right-handed people.

With reference to the first embodiment, a plane 13 along which an upper surface 21 of the functional face 2 extends in parallel to a plane 14 along which the upper surface 42 of the movable part 4 extends in the folded position 5. In practice, the plane 13 along which the upper surface 21 of the functional face 2 extends and the plane 14 along which the upper surface 42 extends, the movable part 4 in the folded position 5, preferably form an angle less than 20° between them. According to the embodiment, the movement of the movable part 4 takes place only in the plane 14. Also, the upper surface 42 of the movable part 4 in the folded position 5 and in the deployed position 6 is included in the plane 14. This allows the user to perform the count in a position in which the wrist is relaxed.

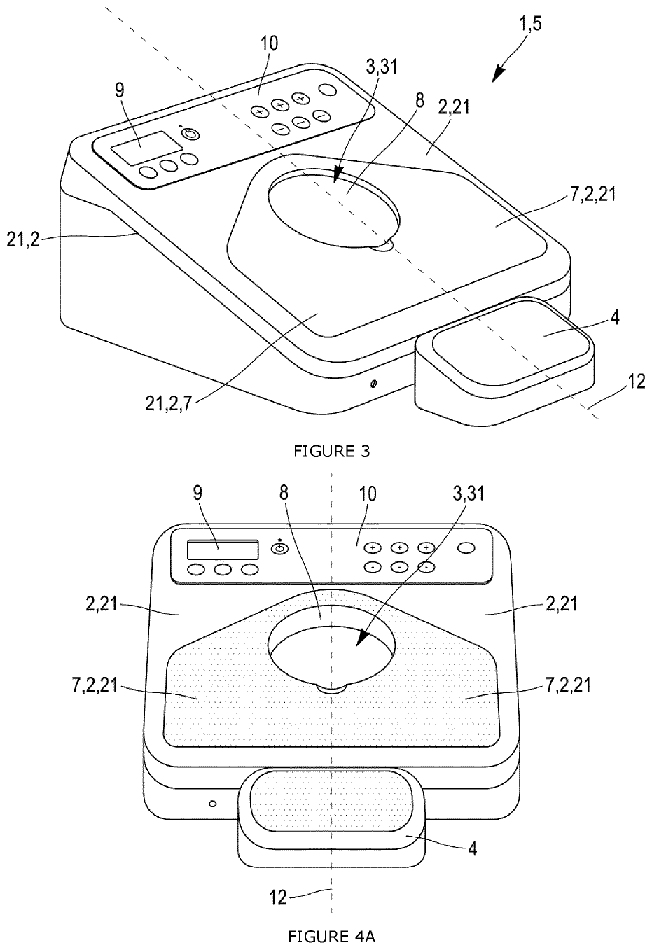

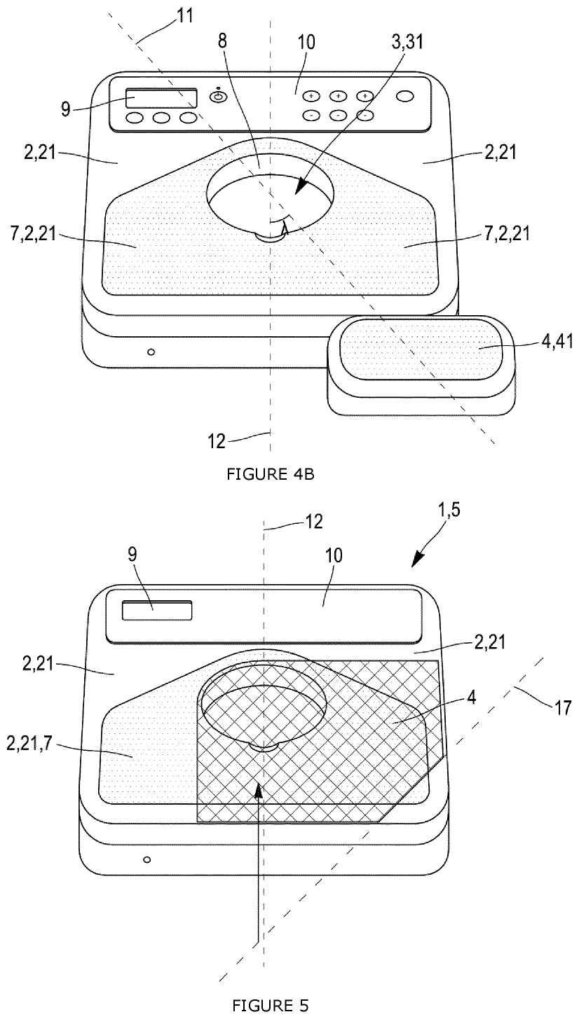

With reference to the second embodiment, and with reference to FIGS. 3 and 4, the moving part 4 is arranged to be translated along the functional face 2. According to the embodiment, and in a non-limiting manner, the moving part 4 is arranged to be translated along the proximal side adjoining the functional face 2. The colony counter 1 according to the first embodiment is suitable for both left-handed and right-handed people.

With reference to the second embodiment, the movable part 4 could be arranged to be translated along one of the lateral sides adjoining the functional face 2. In this case, to suit right-handers and left-handers, the counter colonies 1 preferably comprises a movable part 4 arranged to be translated along one of the two lateral sides adjoining the functional face 2 and a movable part 4 arranged to be translated along the other on the two lateral sides adjoining the functional face 2. With reference to the first and second embodiments, the angle À is adjustable. Indeed, by translating the moving part 4 more or less or by pivoting the moving part 4 more or less, the user will be able to adapt to his morphology the position of the moving part 4 in its deployed position 6 and therefore the value of the angle À.

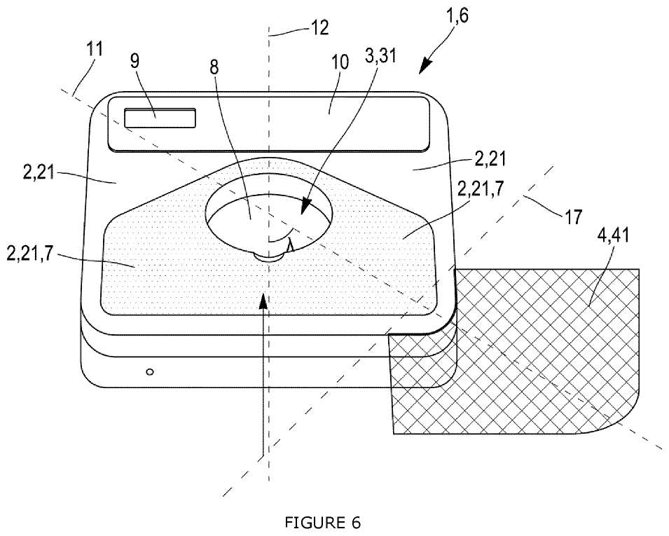

According to the third embodiment, and with reference to FIGS. 5 and 6, the movable part 4 is arranged to pivot around an axis of rotation 17 parallel to the upper surface 21 of the functional face 2. Furthermore, in the position re-folded 5, the movable part 4 covers the counting zone 3. Also, in the re-folded position 5, the movable part 4 is therefore used as a cover protecting the counting zone 3. The colony counter I according to the third mode of realization is suitable for right-handed people. However, the colony counter I can be arranged so that the moving part 4 is removable and arranged to be arranged so as to be suitable for left-handers and right-handers.

With reference to the third embodiment, the plane 13 along which an upper surface 21 of the functional face 2 extends coincides with the plane 14 along which the upper surface 42 of the movable part 4 extends in the deployed position 6. The plane 13 along which an upper surface 21 of the functional face 2 extends is parallel with the plane 14 along which the upper surface 42 of the movable part 4 extends in the folded position 5. In practice, the plane 13 along which extends the upper surface 21 of the functional face 2 and the plane 14 along which the upper surface 42 extends the movable part 4 in the deployed position 6 preferably form an angle less than 20° between them. This allows the user to perform the count in a position in which the wrist is relaxed.

Of course, the invention is not limited to the examples which have just been described and numerous adjustments can be made to these examples without departing from the scope of the invention.

Thus, in variants that can be combined with each other of the previously described embodiments:
in the deployed position 6, the surface 41 of the movable part 4 forms a support surface 41 for the hand and/or the wrist and/or the forearm and/or the elbow of the user, and/or
the additional support surface 7 serves as a hand rest and/or hand wrist rest and/or forearm rest for the user, and/or
as an alternative, the functional face 2 comprises an orifice intended to receive the microorganism culture support and one or more elements extending into the orifice from a periphery of the orifice.

In addition, the different features, shapes, variants and embodiments of the invention can be associated with each other in various combinations as long as they are not incompatible or exclusive of each other.

1. A manual colony counter for counting microorganisms of a culture support for microorganisms, the manual colony counter comprising:
a functional face, wherein the functional face comprises a counting zone on which the culture support for microorganisms to be counted manually can be rested;
a movable part movable to be arranged in at least two different positions relative to the functional face;
wherein the movable part has a deployed position wherein a surface of the movable part forms a support surface for a hand, wrist, forearm, and/or elbow of a user; and
wherein the movable part has a retracted position.

2. The manual colony counter of claim 1, in which wherein, when the movable part is in the deployed position, an axis connecting a center of the support surface to a center of the counting zone forms an angle À of between 20° and 70° relative to a median axis extending between one side of the functional face adapted to be disposed facing the user and the center of the counting zone.

3. The manual colony counter of claim 2, wherein, when the movable part is in the deployed position, the functional face and the movable part form an angle less than or equal to 45° between them.

4. The manual colony counter of claim 1, wherein the manual colony counter has a face on which the manual colony counter is adapted to rest and wherein the functional face forms an angle β of between 12.5° and 27.5° relative to there face is on which the manual colony counter is adapted to rest.

5. The manual colony counter of claim 1, wherein the counting zone comprises:
a recess comprising a bottom on which the microorganism culture support can be rested or
an orifice comprising, on its periphery, one or more gripping means on which is the microorganism culture support can be rested.

6. The manual colony counter of claim 1, wherein, when the movable part is in the retracted position, at least part of the movable part covers the counting zone.

7. The manual colony counter of claim 1, wherein the movable part is arranged to be translated along the functional face or along a side adjoining the functional face.

8. The manual colony counter of claim 1, wherein the movable part is arranged to pivot around an axis of rotation parallel to the functional face or around a axis of rotation perpendicular to the functional face.

9. The manual colony counter of claim 1, further comprising a control unit arranged to:
activate the colony counter in response to a passage of the moving part from the retracted position to the deployed position, and/or
deactivate the colony counter in response to a passage of the moving part from the deployed position to the retracted position.

10. The manual colony counter of claim 1, wherein the bearing surface of the movable part is formed by an elastic material.

11. The manual colony counter of claim 1, further comprising a culture support for microorganisms wherein the culture support for microorganisms comprises a Petri dish.

12. The manual colony counter of claim 3, wherein, when the movable part is in the deployed position, the functional face and the movable part are parallel.
